(19) United States
(12) Patent Application Publication
(10) Pub. No.: US 2022/0164576 A1
(43) Pub. Date: May 26, 2022

(54) SURGICAL INSTRUMENT INVENTORY SYSTEM AND SURGICAL INSTRUMENT INVENTORY METHOD

(71) Applicant: Metal Industries Research & Development Centre, Kaohsiung (TW)

(72) Inventors: Sheng-Hong Yang, Kaohsiung City (TW); Bo-Wei Pan, Kaohsiung City (TW); Jian-Jia Tzeng, Kaohsiung City (TW)

(73) Assignee: Metal Industries Research & Development Centre, Kaohsiung (TW)

(21) Appl. No.: 17/103,957

(22) Filed: Nov. 25, 2020

(51) Int. Cl. G06K 9/00; G06K 9/46; G06K 9/62; G16H 40/20; G06N 3/08

(52) U.S. Cl. CPC G06K 9/00624; G06K 9/46; G06N 3/08; G16H 40/20; G06K 9/6202

(57) ABSTRACT

A surgical instrument inventory system, including a memory and a processor, is provided. The memory is configured to store a global identification module and a local identification module. The processor is coupled to the memory and is configured to input a surgical instrument image to the global identification module and the local identification module. The global identification module is configured to output multiple global image features corresponding to the surgical instrument image. The local identification module is configured to output multiple local image features corresponding to an instrument end image of the surgical instrument image. The processor determines a surgical instrument type of the surgical instrument image according to the multiple global image features and the multiple local image features.

### BACKGROUND

### Technical Field

This disclosure relates to an inventory technology, and in particular to a surgical instrument inventory system and a surgical instrument inventory method.

### Description of Related Art

Currently, during preparation of surgical instruments before an operation, medical personnel have to prepare the various surgical instruments required for the operation before the operation. For example, the medical personnel have to store multiple surgical instruments in a surgical instrument tray or a surgical instrument pack, and perform an inventory of the surgical instruments manually. In other words, for different operations, the medical personnel have to spend a lot of time preparing and inventorying the surgical instruments for each operation. Therefore, the conventional preparation and inventory of the surgical instruments are prone to human error, such as occurrence of incorrectly placed surgical instruments or missing surgical instruments. In view of this, several embodiments detailing how to automatically and accurately inventory the surgical instruments are proposed as follows.

### SUMMARY

This disclosure provides a surgical instrument inventory system and a surgical instrument inventory method that can provide the function of automatically inventorying a surgical instrument.

A surgical instrument inventory system of the disclosure includes a memory and a processor. The memory is configured to store a global identification module and a local identification module. The processor is coupled to the memory and is configured to input a surgical instrument image to the global identification module and the local identification module. The global identification module is configured to output multiple global image features corresponding to the surgical instrument image. The local identification module is configured to output multiple local image features corresponding to an instrument end image of the surgical instrument image. The processor determines a surgical instrument type of the surgical instrument image according to the multiple global image features and the multiple local image features.

A surgical instrument inventory method of the disclosure includes the following steps. A surgical instrument image is inputted to a global identification module to generate multiple global image features corresponding to the surgical instrument image. The surgical instrument image is inputted to a local identification module to generate multiple local image features corresponding to an instrument end image of the surgical instrument image. A surgical instrument type of the surgical instrument image is determined based on the multiple global image features and the multiple local image features.

Based on the above, the surgical instrument inventory system and the surgical instrument inventory method of the disclosure may identify the type of the surgical instrument by means of image analysis, so as to accurately perform category inventory and quantity counting of the surgical instrument.

To make the abovementioned features and advantages of the disclosure more comprehensible, exemplary embodiments in concert with drawings are described in detail below.

### BRIEF DESCRIPTION OF THE DRAWINGS

FIG. 1 is a schematic diagram of a surgical instrument inventory system according to an embodiment of the disclosure.

FIG. 2 is a flowchart of obtaining a surgical instrument image according to an embodiment of the disclosure.

FIG. 3 is a projection schematic diagram of a virtual surgical instrument tray image according to an embodiment of the disclosure.

FIG. 4 is a flowchart of a surgical instrument inventory method according to an embodiment of the disclosure.

FIG. 5 is a schematic diagram of generating multiple image features corresponding to the surgical instrument image according to an embodiment of the disclosure.

### DESCRIPTION OF THE EMBODIMENTS

In order to make the content of the disclosure more comprehensible, the following embodiments are specifically cited as examples in which the disclosure may be implemented. In addition, wherever possible, elements/members/steps with the same reference numerals in the drawings and implementation means represent the same or similar components.

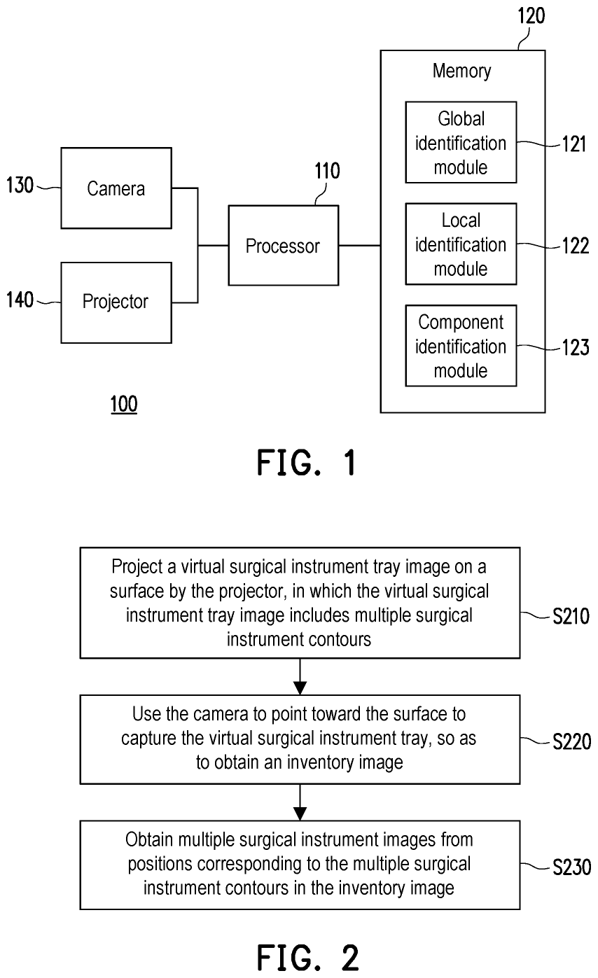

FIG. 1 is a schematic diagram of a surgical instrument inventory system according to an embodiment of the disclosure. With reference to FIG. 1, a surgical instrument inventory system 100 includes a processor 110, a memory 120, a camera 130, and a projector 140. The processor 110 is coupled to the memory 120, the camera 130, and the projector 140. In the embodiment, the memory 120 may store a global identification module 121, a local identification module 122, and a component identification module 123. In the embodiment, the surgical instrument inventory system 100 may first use the projector 140 to project a virtual surgical instrument tray image on a surface. The virtual surgical instrument tray image may include one or more surgical instrument contours, and the multiple surgical instrument contours may correspond to a same or different surgical instrument types. In the embodiment, after a user places a corresponding surgical instrument on the virtual surgical instrument tray image, the surgical instrument inventory system 100 may then use the camera 130 to capture the virtual surgical instrument tray image to obtain an inventory image. The inventory image may include one or more surgical instrument images.

In the embodiment, the processor 110 may analyze the one or more surgical instrument images in the inventory image through the global identification module 121, the local identification module 122, and the component identification module 123, so as to generate multiple image features corresponding to each of the surgical instrument images. The processor 110 may effectively respectively identify a surgical instrument type of the corresponding surgical instrument image according to the multiple image features. In addition, after the processor 110 determines multiple surgical instrument types of the multiple surgical instrument images in the same inventory image, the processor 110 may decide on inventory information of the corresponding inventory image based on the identified multiple surgical instrument types of the multiple surgical instruments. In addition, the processor 110 may also, for example, calculate the number of the same or different types of surgical instruments, and record them in the inventory information.

In some embodiments of the disclosure, the surgical instrument inventory system 100 may not include the projector 140, but may still achieve the function of automatically inventorying the surgical instrument according to the disclosure. In this regard, in some embodiments of the disclosure, the user may place the corresponding surgical instrument on a physical surgical instrument tray or the virtual surgical instrument tray image projected by another projection equipment, to enable the surgical instrument inventory system 100 to use the camera 130 to capture the physical surgical instrument tray or the virtual surgical instrument tray image projected by the another projection equipment, so as to obtain the inventory image. Alternatively, in other implementation scenarios of the disclosure, the user may place one or more of the surgical instruments on any platform for the surgical instrument inventory system 100 to perform the identification and inventory of the surgical instruments.

In other embodiments of the disclosure, the surgical instrument inventory system 100 may not include the camera 130, but may still achieve the function of automatically inventorying the surgical instrument of the disclosure. In this regard, in other embodiments of the disclosure, the user may obtain the inventory image through any independent photography equipment. The user may input the inventory image to the processor 110 of the surgical instrument inventory system 100 to perform related image processing and analysis calculations as proposed in the various embodiments of the disclosure.

In some other embodiments of the disclosure, the memory 120 of the surgical instrument inventory system 100 may only store the global identification module 121 and the local identification module 122. In this regard, in the some other embodiments of the disclosure, the processor 110 of the surgical instrument inventory system 100 may only use the global identification module 121 and the local identification module 122 to analyze the one or more surgical instrument images in the inventory image, so as to generate the multiple image features corresponding to the each of the surgical instrument images. In this regard, although identification accuracy of the embodiment is different from identification accuracy of the embodiment including the component identification module 123, in the some other embodiments (for example, in applied scenarios where there is a larger variation in the surgical instrument types between the multiple surgical instruments), the processor 110 may still effectively respectively identify the surgical instrument type of the corresponding surgical instrument image according to the multiple image features.

In the embodiment, the processor 110 may include a central processing unit (CPU), a programmable general-purpose or special-purpose microprocessor, a digital signal processor (DSP), a programmable controller, an application specific integrated circuit (ASIC), a graphics processing unit (GPU), or other similar elements, or a combination of the above elements, and may be configured to implement related functional circuits of the disclosure.

In the embodiment, the memory 120 may include, for example, a random-access memory (RAM), a read-only memory (ROM), an optical disc, a magnetic disk, a hard drive, a solid-state drive, a flash drive, a security digital (SD) card, a memory stick, a compact flash (CF) card, or any type of storage device. In the embodiment, the memory 120 may be configured to store the related modules, related image data, and related parameters mentioned in the various embodiments of the disclosure, to enable the processor 110 to access the memory 120 to execute related data processing and calculation.

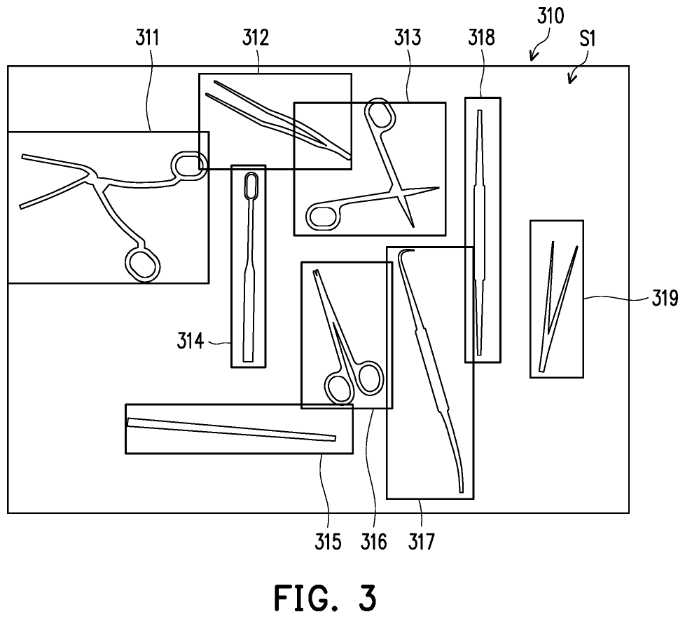

FIG. 2 is a flowchart of obtaining a surgical instrument image according to an embodiment of the disclosure. FIG. 3 is a projection schematic diagram of a virtual surgical instrument tray image according to an embodiment of the disclosure. With reference to FIGS. 1 to 3, the surgical instrument inventory system 100 may execute Steps S210 to S230 as shown in FIG. 2 to obtain the surgical instrument image, and the surgical instrument inventory system 100 may project a virtual surgical instrument tray image 310 as shown in FIG. 3. In the Step S210, the surgical instrument inventory system 100 may project the virtual surgical instrument tray image 310 on a surface 51 by the projector 140. The virtual surgical instrument tray image 310 includes multiple surgical instrument contours 311 to 319. In the embodiment, the surface 51 may refer to the surgical instrument tray or a surgical instrument pack, but the disclosure is not limited thereto. In some implementation scenarios of the disclosure, the surface 51 may refer to a surface of any platform (which may be a flat surface or a non-flat surface), and the disclosure does not limit projection position and projection environment. In the embodiment, the user may place the corresponding multiple surgical instruments on the surgical instrument contours 311 to 319 in the virtual surgical instrument tray image 310. After the user has completed the placement, the user may operate the surgical instrument inventory system 100 to perform image-taking or the surgical instrument inventory system 100 may automatically image-taking at a preset time. Alternatively, the surgical instrument inventory system 100 may pre-take an image to determine whether there are objects in positions corresponding to the surgical instrument contours 311 to 319 in the obtained image by means of image analysis, so as to perform the image-taking operation of the inventory image.

It is worth noting that the virtual surgical instrument tray image 310 may help the user to place correct number of and correct surgical instrument type of the one or more surgical instruments on one or more specific positions on the surgical instrument tray, so as to conform to current surgical medical equipment specifications, or to facilitate correct and convenient retrieval and usage by the medical personnel during the operation. However, in some implementation scenarios of the disclosure, the surgical instrument inventory system 100 may also directly execute Step S220 as follows, without having to execute the Step S210.

In the Step S220, the surgical instrument inventory system 100 may use the camera 130 to point toward the surface to capture the virtual surgical instrument tray 310, so as to obtain the inventory image. In Step S230, the surgical instrument inventory system 100 may obtain the multiple surgical instrument images from the positions corresponding to the multiple surgical instrument contours in the inventory image. Therefore, the surgical instrument inventory system 100 may perform surgical instrument identification as in the following embodiments for each of the acquired multiple surgical instrument images. In addition, it should be noted that projection image of the virtual surgical instrument tray of the disclosure is not limited to that shown in FIG. 3, and the number of the surgical instruments and the types of the surgical instruments in the virtual surgical instrument tray are not limited to those shown in FIG. 3.

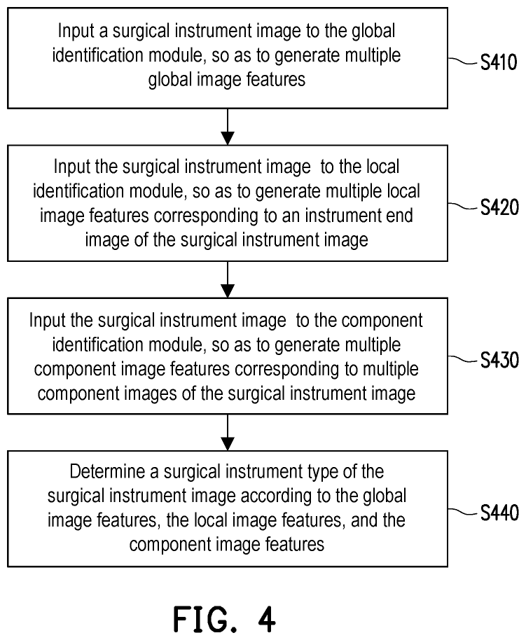

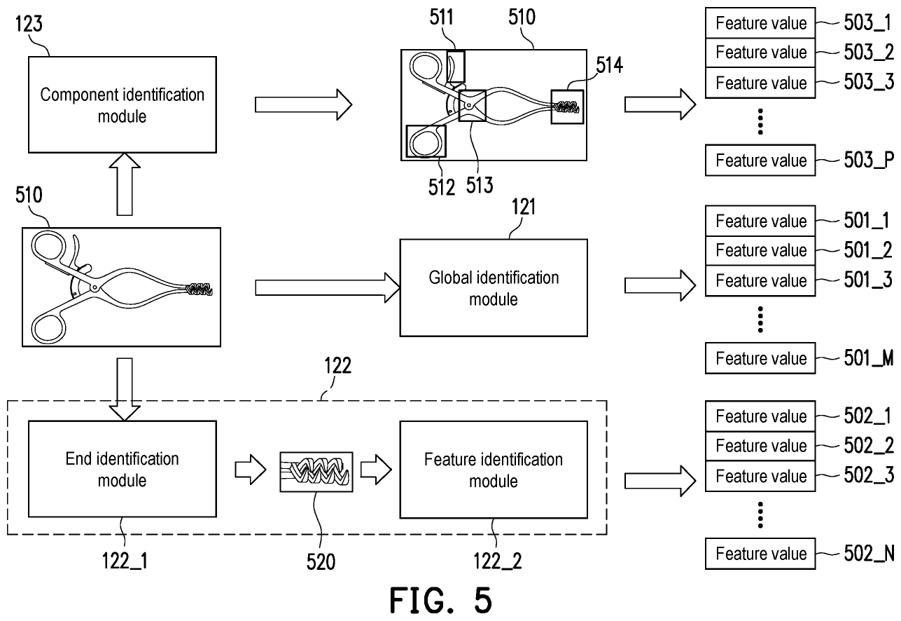

FIG. 4 is a flowchart of a surgical instrument inventory method according to an embodiment of the disclosure. FIG. 5 is a schematic diagram of generating multiple image features corresponding to the surgical instrument image according to an embodiment of the disclosure. With reference to FIGS. 1, 4 and 5, the surgical instrument inventory system 100 may execute steps S410 to S440 as shown in FIG. 4 to determine the surgical instrument type of the surgical instrument, and results of multiple modules executed by the processor 110 of the surgical instrument inventory system 100 may be as shown in FIG. 5. In the Step S410, the processor 110 of the surgical instrument inventory system 100 may input a surgical instrument image 510 to the global identification module 121, so as to generate multiple global image features 501_1 to 501_M (respectively having different feature values) corresponding to the surgical instrument image 510, where M is a positive integer. In the embodiment, the surgical instrument image 510 may be, for example, one of the multiple surgical instrument contours 311 to 319 taken from the virtual surgical instrument tray image 310 of the embodiment in FIG. 3. In the embodiment, the global identification module 121 is configured to perform convolutional neural network (CNN) calculation on the surgical instrument image 510, so as to output the global image features 501_1 to 501_M.

In Step S420, the processor 110 of the surgical instrument inventory system 100 may input the surgical instrument image 510 to the local identification module 122, so as to generate multiple local image features 502_1 to 502_N (respectively having different feature values) corresponding to an instrument end image 520 of the surgical instrument image 510, where N is a positive integer. In the embodiment, the local identification module 122 may further include an end identification module 122_1 and a feature identification module 122_2. The local identification module 122 may input the surgical instrument image 510 to the end identification module 122_1, to enable the end identification module 122_1 to perform Faster R-CNN to decide on the instrument end image 520 from the surgical instrument image 510. Image size of the instrument end image 520 is smaller than that of the surgical instrument image 510. Then, the local identification module 122 may input the local identification module 122 to the feature identification module 122_2, to enable the feature identification module 122_2 to perform the convolutional neural network calculation, so as to output the local image features 502_1 to 502_N corresponding to the instrument end image 520. In other words, since the surgical instruments of the different surgical instrument types may have different instrument end structures, the surgical instrument inventory system 100 of the embodiment may help to distinguish the different surgical instruments by identifying the instrument end structures of the surgical instruments.

In Step S430, the processor 110 of the surgical instrument inventory system 100 may input the surgical instrument image 510 to the component identification module 123, so as to generate multiple component image features 503_1 to 503_P corresponding to multiple component images 511 to 514 of the surgical instrument image 510 (respectively having different feature values), where P is a positive integer. In the embodiment, the component identification module 123 is configured to perform fast convolutional neural network calculation on the surgical instrument image 510 to decide on the component images 511 to 514 from the surgical instrument image 510, and respectively perform components analysis on the component images 511 to 514, so as to decide on the component image features 503_1 to 503_P corresponding to the component images 511 to 514. In this regard, since the surgical instruments of the different surgical instrument types may have different component structures, the surgical instrument inventory system 100 of the embodiment may help to distinguish the different surgical instruments by identifying at least one specific component structure of the surgical instrument.

In the Step S440, the processor 110 of surgical instrument inventory system 100 may determine the surgical instrument type of the surgical instrument image 510 according to the global image features 501_1 to 501_M, the local image features 502_1 to 502_N, and the component image features 503_1 to 503_P. In this regard, since the surgical instruments of the different surgical instrument types may have the different overall (global) instrument image features, instrument end structures, and component structures, the surgical instrument inventory system 100 of the embodiment may identify the surgical instrument type of the surgical instrument by obtaining the multiple image features from image recognition results. In addition, the surgical instrument inventory system 100 of the embodiment may store an instrument comparison table in, for example, the memory 120. The instrument comparison table may, for example, store multiple preset global image feature values, local preset image feature values, and component preset image feature values of each of the one or more surgical instruments. Therefore, the processor 110 may compare the global image features 501_1 to 501_M, the local image features 502_1 to 502_N and the component image features 503_1 to 503_P with the instrument comparison table (whether the values are consistent with the preset feature values), so as to quickly and accurately determine the surgical instrument type of the surgical instrument image 510.

Moreover, in the embodiment, the memory 120 is further configured to store an inventory comparison table. The processor 110 may, for example, compare information of the multiple surgical instrument types with the inventory comparison table, so as to decide on inventory information, after the information of the corresponding multiple surgical instrument types is obtained when the surgical instrument inventory system 100 performs identification operation of the Steps S410 to S440 on the multiple surgical instrument images corresponding to the positions of the surgical instrument contours 311 to 319 of the captured virtual surgical tray 310 in FIG. 3. For example, the inventory comparison table includes a scissors, a plier, and a syringe. In this regard, if the surgical instrument inventory system 100 is able to determine the surgical instrument image having the scissors, the plier, and the syringe from the same inventory image, it means that the number and the type of the surgical instruments placed on the surgical instrument tray by the user are correct. Therefore, the surgical instrument inventory system 100 of the embodiment can quickly and accurately perform inventory of the surgical instruments automatically.

In addition, it is worth noting that the global identification module 121, the local identification module 122, and the component identification module 123 may be pre-trained to realize their image identification and analysis functions. In this regard, the user may capture multiple reference surgical instrument images (for example, 100) in advance, and input the multiple reference surgical instrument images having a BBOX list composed of all target marking boxes in the images into the above-mentioned convolutional neural network calculation model or fast convolutional neural network calculation model of the global identification module 121, the local identification module 122, and the component recognition module 123 for training and inference, to enable the convolutional neural network calculation model or fast convolutional neural network calculation model of the global identification module 121, the local identification module 122 and component identification module 123 to correspondingly output multiple identification results. Then, after data conversion (such as converting the BBOX list into a user-editable Pascal VOC XML format), the user may amend the BBOX list of each of the reference surgical instrument images in the multiple identifications results, and then re-input the multiple reference surgical instrument images having the amended BBOX list to the above-mentioned convolutional neural network calculation model or fast convolutional neural network calculation model of the global identification module 121, the local identification module 122, and the component identification module 123 for training. In this regard, the above-mentioned convolutional neural network calculation model or fast convolutional neural network calculation model of the global identification module 121, the local identification module 122, and the component identification module 123 may be, for example, trained through multiple cycles and continuously adding multiple references surgical instrument images (such as, adding 1000 pieces) to enable the above-mentioned global identification module 121, the local identification module 122 and the component identification module 123 to accurately and effectively identify the global image features, the global image features and the component image features.

In summary, the surgical instrument inventory system and the surgical instrument inventory method of the disclosure can quickly and accurately identify the surgical instruments through means such as multi-view image analysis and the neural network calculations, and can accurately and automatically inventory the type and the number of the multiple surgical instruments through look-up tables.

In order to make the content of the disclosure more comprehensible, the following embodiments are specifically cited as examples in which the disclosure may be implemented. In addition, wherever possible, elements/members/steps with the same reference numerals in the drawings and implementation means represent the same or similar components.

What is claimed is:

1. A surgical instrument inventory system, comprising:
a memory, configured to store a global identification module and a local identification module; and
a processor, coupled to the memory, and is configured to input a surgical instrument image to the global identification module and the local identification module,
wherein the global identification module is configured to output a plurality of global image features corresponding to the surgical instrument image, and the local identification module is configured to output a plurality of local image features corresponding to an instrument end image of the surgical instrument image,
wherein the processor determines a surgical instrument type of the surgical instrument image according to the global image features and the local image features.

2. The surgical instrument inventory system according to claim 1, wherein the global identification module and the local identification module are respectively configured to perform convolutional neural network (CNN) calculation on the surgical instrument image and the instrument end image, so as to output the global image features and the local image features, wherein image size of the instrument end image is smaller than that of the surgical instrument image, wherein the local identification module is also configured to perform Faster R-CNN calculation on the surgical instrument image, so as to decide on the instrument end image from the surgical instrument image.

3. The surgical instrument inventory system according to claim 1, wherein the memory is further configured to store a component identification module, and the processor is further configured to input the surgical instrument image to the component identification module,
wherein the component identification module is configured to output a plurality of component image features of a plurality of component images corresponding to the surgical instrument image, and the processor determines the surgical instrument type of the surgical instrument image according to the global image features, the global image features, and the component image features,
wherein the component identification module is configured to perform Faster R-CNN calculation on the surgical instrument image, so as to decide on the component images from the surgical instrument image,
wherein the component identification module is further configured to perform component analysis on the component images to decide on the component image features corresponding to the component images.

4. The surgical instrument inventory system according to claim 1, further comprising:
a projector, coupled to the processor, and is configured to project a virtual surgical instrument tray image on a surface, wherein the virtual surgical instrument tray image comprises a plurality of surgical instrument contours; and
a camera, coupled to the processor, and is configured to point toward the surface to capture the virtual surgical instrument tray, so as to obtain an inventory image, wherein the inventory image comprises a plurality of surgical instrument images,
wherein the processor obtains the surgical instrument images from positions corresponding to the surgical instrument contours in the inventory image, and the processor respectively identifies the surgical instrument types of the surgical instrument images,
wherein the memory is further configured to store an inventory comparison table, and the processor compares the surgical instrument types with the inventory comparison table, so as to decide on inventory information.

5. The surgical instrument inventory system according to claim 1, wherein the memory is further configured to store an instrument comparison table, and the processor compares the global image features and the local image features with the instrument comparison table to determine the surgical instrument type of the surgical instrument image.

6. A surgical instrument inventory method, comprising:
inputting a surgical instrument image to a global identification module to generate a plurality of global image features corresponding to the surgical instrument image;
inputting the surgical instrument image to a local identification module to generate a plurality of local image features corresponding to an instrument end image of the surgical instrument image; and
determining a surgical instrument type of the surgical instrument image according to the global image features and the local image features.

7. The surgical instrument inventory method according to claim 6, wherein the global identification module and the local identification module are respectively configured to perform convolutional neural network (CNN) calculation on the surgical instrument image and the instrument end image, so as to output the global image features and the local image features, wherein image size of the instrument end image is smaller than that of the surgical instrument image, wherein the local identification module is also configured to perform Faster R-CNN calculation on the surgical instrument image, so as to decide on the instrument end image from the surgical instrument image.

8. The surgical instrument inventory method according to claim 6, further comprising:
inputting the surgical instrument image to a component identification module to generate a plurality of component image features corresponding to a plurality of component images of the surgical instrument image; and
determining the surgical instrument type of the surgical instrument image according to the global image features, the local image features, and the component image features,
wherein the component identification module is configured to perform Faster R-CNN calculation on the surgical instrument image, so as to decide on the component images from the surgical instrument image,
wherein the component identification module is further configured to perform component analysis on the component images to decide on the component image features corresponding to the component images.

9. The surgical instrument inventory method according to claim 6, further comprising:
projecting a virtual surgical instrument tray image on a surface by a projector, wherein the virtual surgical instrument tray image comprises a plurality of surgical instrument contours;
pointing toward the surface to capture the virtual surgical instrument tray by a camera, so as to obtain an inventory image, wherein the inventory image comprises a plurality of surgical instrument images;
obtaining the surgical instrument images from positions corresponding to the surgical instrument contours in the inventory image; and
respectively identifying the surgical instrument types of the surgical instrument images, and comparing the surgical instrument types with an inventory comparison table, so as to decide on inventory information corresponding to the inventory image.

10. The surgical instrument inventory method according to claim 6, wherein determination of the surgical instrument type of the surgical instrument image comprises:
comparing the global image features and the global image features with an instrument comparison table, so as to determine the surgical instrument type of the surgical instrument image.
